(19) United States
(12) Patent Application Publication
(10) Pub. No.: US 2024/0382345 A1
(43) Pub. Date: Nov. 21, 2024

(54) TOPICAL PAIN-RELIEF DEVICES HAVINGA BROKEN CONDUCTIVE LAYER

(71) Applicant: Leonard Albert Lee, Orem, UT (US)

(72) Inventor: Leonard Albert Lee, Orem, UT (US)

(21) Appl. No.: 18/669,506

(22) Filed: May 20, 2024

Related U.S. Application Data
(60) Provisional application No. 63/467,907, filed on May 19, 2023.

(51) Int. Cl. A61F 13/02; A61F 13/00; A61L 15/32; A61L 15/58

(52) U.S. Cl. CPC A61F 13/0233; A61L 15/32; A61L 15/58; A61F 2013/00259

(57) ABSTRACT

A pain-relief device for treating pain related to soft tissue injuries and nerve pain has a broken and at least partially interconnected, conductive layer made of metal particles which are screen printed on a flexible substrate. No attempt is made to electrically insulate any of the metal particles from adjacent particles. The device can be configured either as a pain-relief patch, or as a pain-relief temporary tattoo. Both the patch and the temporary tattoo are adhered to a patient's skin at a site of pain with a non-allergenic adhesive. The flexible substrate for the pain-relief patch is a polymeric material such as polyester-based thermoplastic polyurethane. The flexible substrate for the temporary tattoo is gum arabic coated decal paper that can be removed after the temporary tattoo is adhered to the patient's skin.

### RELATED APPLICATIONS

This application has a priority date of May 19, 2023, based on the filing of provisional patent application 63/467,907 on that date.

### BACKGROUND OF THE INVENTION

### Field of the Invention

This invention relates, generally, to topical compositions and, more particularly, to pain-relief devices covered with a broken conductive pattern for treating pain resulting from injuries and diseases in animals, including humans.

### DESCRIPTION OF THE PRIOR ART

The use of metals as topical dressings has been proposed by numerous individuals. Rhett Francis Spencer and Anthony Joseph Sutera, in U.S. Pat. Nos. 10,707,570 and 11,380,985, have proposed the use of a metallic layer in a patch that is applied to the body at a location corresponding to a source of pain or at a location corresponding to the source of pain and the brain. The metallic layer, which the inventors term “a reactive capacitance material”, is sandwiched between a pair of non-conductive layers, and comprises conductive particles dispersed in a dielectric binder so that at least a majority of the conductive particles are adjacent, but do not touch, one another. It is unclear just what the term reactive capacitance material means, as in order to have capacitance, there must be two parallel plates and a voltage source to oppositely charge the two plates. Because most of the metallic particles in the reactive capacitive layer are electrically isolated from one another, it is unclear how the layer could act as a capacitive plate, as it could not be charged. Reactance, on the other hand, is the opposition to the flow of alternating current caused by inductance and capacitance in a circuit rather than by resistance. Though there is certainly currently flow in the human body and that of animals, one would be hard pressed to catagorize such current flow as alternating current.

U.S. Pat. No. 10,195,148, by Gary Karpf, describes ointments for topical use comprising elemental metal particles distributed within an insulating matrix such as petroleum jelly, lanolin, silicone, wax, or combinations thereof, wherein the ointments, which are non-conducting, impart capacitance to the composition which alters electrostatic and electromagnetic fields. Given the fact that the ointments are non-conductive, it is unclear how such compositions could have the attribute of capacitance, or the ability to store an electrical charge.

### SUMMARY OF THE INVENTION

The present invention provides devices having a broken conductive pattern for treating pain related to soft tissue injuries and diseases. There are two primary embodiments of those devices. The term broken conductive pattern means, that instead of being an unbroken layer of uniform thickness, portions of the layer are missing throughout, such that multiple, interconnected conductive traces, or paths, are formed throughout the layer.

The first embodiment device is a pain-relief patch that is fabricated by screen printing the broken, yet at least partially interconnected, layer of conductive particles mixed with a polymeric carrier liquid and volatile solvent on a flexible polymeric substrate. No attempt is made to electrically isolate any of the conductive particles from adjacent particles. Thus, the screen-printed conductive particles, following evaporation of the volatile solvent, function as a conductive layer, with gaps in the conductive layer where no conductive particles are deposited during the screen printing process. Silver coated copper particles and powdered graphite are two types of conductive particles that have been successfully used for this application. The entire layer of conductive particles is then optionally covered by an insulative layer which provides additional durability to the conductive particle layer. A thin non-allergenic adhesive layer is then applied over the conductive particle layer and optional insulative layer so that the patch can be adhered to the skin at a site of pain on a wearer's body. Silicone adhesives are presently preferred for this application.

The second embodiment of the invention uses a pain patient's skin as a flexible substrate on which a broken, yet at least partially interconnected, layer of conductive particles is adhesively applied in much the same manner that a temporary tattoo is applied to one's skin. The broken conductive pattern is screen printed using the same techniques employed for the first embodiment of the invention. That is to say that conductive particles, such as silver-coated copper particles or powdered graphite particles are mixed with a polymeric carrier liquid and volatile solvent are screen printed. However, instead of printing the conductive pattern on a flexible polymeric substrate, the conductive pattern is screen printed on decal backing paper that has been coated with a gum arabic layer. Gum arabic is a complex mixture of glycoproteins and polysaccharides, predominantly polymers of arabinose and galactose. It is soluble in water, edible, and used primarily in the food industry and soft-drink industry as a stabilizer, with E number E414 (I414 in the US). Gum arabic is a key ingredient in traditional lithography and is used in printing, paints, glues, cosmetics, and various industrial applications, including viscosity control in inks and in textile industries, though less expensive materials compete with it for many of these roles. A solvent-based, breathable, nonalergenic adhesive layer is then screen printed on top of the conductive pattern. Once the adhesive layer is dry, a release film, such as 1 mil film of polyethylene terephthalate (PET) is applied over the adhesive layer, thereby creating a temporary broken conductive pattern tattoo. In order to apply the temporary broken conductive pattern tattoo to a patient's skin, the release film is peeled away from the adhesive layer, and the temporary tattoo, minus the release film, is applied to the patient's skin at a site of pain. Once, the adhesive layer had adhered to the skin, the decal backing paper may be removed by moistening it, thereby solvating the gum arabic layer, thereby allowing the decal backing paper to be separated from the broken conductive pattern layer. The temporary broken conductive pattern tattoo can remain in place for up to several weeks. The temporary broken conductive pattern layer tattoo can be removed by washing it with warm water and mineral oil.

It is presently not fully understood why the patches are effective at eliminating or mitigating pain. One theory is that the weak electric current in nerve tissue at the site of pain creates magnetic fields, which though also weak, can still interact with the metal layer of the patches through electromagnetic induction, thereby generating eddy currents in the conductive particle layer which are dissipated by the resistance to current flow within the conductive particle layer, itself. The electromagnetic induction and subsequent dissipation of the eddy currents in the conductive particle layer thereby interfere with electrical pain signals being sent to the brain by the body's nervous system. Another theory is that the conductive, broken layer, made of deposited conductive particles, acts as a first capacitor plate. A portion of a pain patient's body over which the pain patch lies, acts as a second capacitor plate. The adhesive layer, as well as the optional insulative layer printed on top of the conductive layer, provides an insulated gap between the first and second plates that creates the capacitor. If an electric charge differential exists between the two plates, electric field strength is certainly not uniform, as field strength presumably drops to zero where there are gaps in the conductive layer. It is possible that this uneven electrical field between the first and second capacitor plates somehow interferes with either the production of electrical pain signals, the attenuation of generated electrical pain signals, or the transmission of electrical pain signals that are produced by the body.

### BRIEF DESCRIPTION OF THE DRAWINGS

FIG. 1 is a bottom plan view of a first iteration of a first embodiment metalized pain-relief device;

FIG. 2 is a bottom plan view of a second iteration of a first embodiment metalized pain-relief device;

FIG. 3 is a bottom plan view of a third iteration of a first embodiment metalized pain-relief device;

FIG. 4 is a top plan view of the third iteration of the first embodiment metalized pain-relief device;

FIG. 5 is a bottom plan view of a fourth iteration of the first embodiment metalized pain-relief device;

FIG. 6 is a bottom plan view of a fifth iteration of the first embodiment metalized pain-relief device;

FIG. 7 is a bottom plan view of a sixth iteration of the first embodiment metalized pain-relief device;

FIG. 8 is bottom plan view of one iteration of a second embodiment metalized pain-relief device; and

FIG. 9 is a ultra-thin slice taken through section line 9-9 of FIG. 8.

### DETAILED DESCRIPTION OF THE INVENTION

The present invention provides devices having a broken conductive pattern for treating pain related to soft tissue injuries and diseases. There are two primary embodiments of those devices. The term broken conductive pattern means, that instead of being an unbroken layer of uniform thickness, portions of the layer are missing throughout, such that multiple, interconnected conductive traces, or paths, are formed throughout the layer.

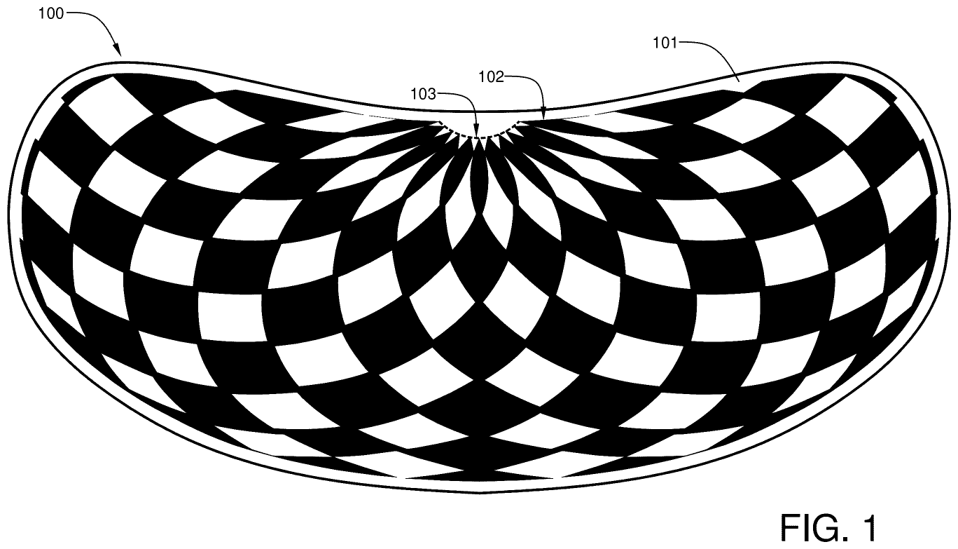

The first embodiment device, which is described with reference to drawing FIGS. 1 through 7, is a pain-relief patch that is fabricated by screen printing the broken, yet at least partially interconnected, layer of conductive particles mixed with a polymeric carrier liquid and volatile solvent on a flexible polymeric substrate. No attempt is made to electrically isolate any of the conductive particles from adjacent particles. Thus, the screen-printed conductive particles, following evaporation of the volatile solvent, function as a conductive layer, with gaps in the conductive layer where no conductive particles are deposited during the screen printing process. Silver coated copper particles and powdered graphite are two types of conductive particles that have been successfully used for this application. The entire layer of conductive particles is then optionally covered by an insulative layer which provides additional durability to the conductive particle layer. A thin non-allergenic adhesive layer is then applied over the conductive particle layer and optional insulative layer so that the patch can be adhered to the skin at a site of pain on a wearer's body. Silicone adhesives are presently preferred for this application.

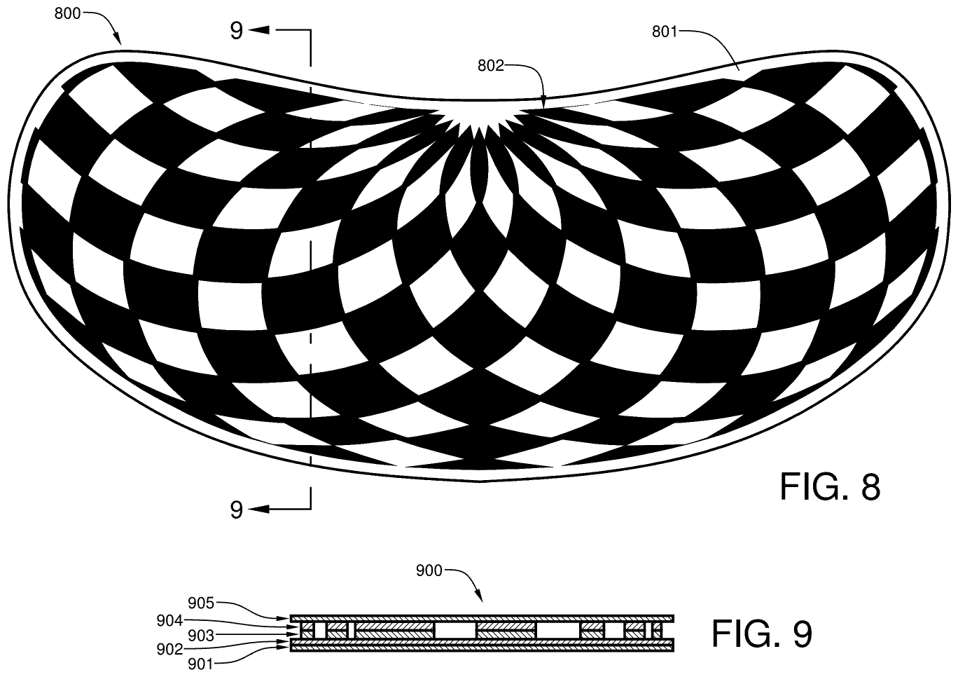

The second embodiment of the invention, which is described with reference to drawing FIGS. 8 and 9, uses a pain patient's skin as a flexible substrate on which a broken, yet at least partially interconnected, layer of conductive particles is adhesively applied in much the same manner that a temporary tattoo is applied to one's skin. The broken conductive pattern is screen printed using the same techniques employed for the first embodiment of the invention. That is to say that conductive particles, such as silver-coated copper particles or powdered graphite particles are mixed with a polymeric carrier liquid and volatile solvent are screen printed. However, instead of printing the conductive pattern on a flexible polymeric substrate, the conductive pattern is screen printed on decal backing paper that has been coated with a water-soluble gum arabic layer. A solvent-based, breathable, nonalergenic adhesive layer is then screen printed on top of the conductive pattern. Once the adhesive layer is dry, a release film, such as 1 mil film of polyethylene terephthalate (PET) is applied over the adhesive layer, thereby creating a temporary broken conductive pattern tattoo. In order to apply the temporary broken conductive pattern tattoo to a patient's skin, the release film is peeled away from the adhesive layer, and the temporary tattoo, minus the release film, is applied to the patient's skin at a site of pain. Once, the adhesive layer had adhered to the skin, the decal backing paper may be removed by moistening it, thereby solvating the gum arabic layer, thereby allowing the decal backing paper to be separated from the broken conductive pattern layer. The temporary broken conductive pattern tattoo can remain in place for up to several weeks. The temporary broken conductive pattern layer tattoo can be removed by washing it with warm water and mineral oil.

In accordance with the first embodiment of the present invention, metalized pain-relief patches for treating pain related to injuries and diseases are fabricating by screen printing a broken, yet completely interconnected layer of conductive particles, such as silver-coated copper particles or powdered graphite particles on a flexible polymeric substrate. No attempt is made to electrically isolate any of the conductive particles from adjacent particles.

Preferred embodiments of the pain-relief patches for treating painful injuries are fabricated preferably using polyester-based thermoplastic polyurethane (TPU). Polyester-based TPU is an inherently soft, tough, versatile elastomer, with excellent drape qualities, which generally requires no plasticizers. These characteristics make polyester-based TPU ideal for many applications. Indentation and rebound hardness of polyester-based TPU remains relatively constant over a wide temperature range for extended periods of time. Not only is polyester-based TPU easily formed and fabricated using conventional methods, it can be welded with radio frequency energy.

The pain-relief patches are fabricating by screen printing a broken, yet at least partially interconnected, patterned layer of conductive particles on a flexible polymeric substrate. No attempt is made to electrically isolate any of the conductive particles from adjacent conductive particles. Silver coated copper particles or powdered graphite particles are screen printed on a polyester-based TPU substrate in a pattern using screen having a mesh within a range of about 120 to about 420 threads per inch (about 47 to about 165 threads per centimeter). The coarser screens, which are preferred, provide patterns of greater metal density in the printed regions. The ink used to print the substrate contains sliver coated copper particles which are about 95 percent copper and about 5 percent silver by composition and having a diameter ranging from 5 to 13×10⁻⁶ m (i.e., 5-13 microns or 1.923 to 5×10⁻⁴ inches). The powdered graphite particles are deemed to have an average diameter within that same range. Although powdered copper can be used in place of the silver-coated copper, it is much more abrasive than the silver-coated copper, and tends to wear out the printing screens rapidly. The ink also contains a polymeric binder compound, as well as a volatile solvent. Excluding the border, about 30 to 50 percent of the substrate remains bare (i.e., unprinted) to minimize deformation of the substrate following screen printing, as the substrate absorbs some of the volatile solvent, which causes the substrate to temporarily swell. As the solvent evaporates from the substrate, the substrate returns to its original planar configuration. Pain-relief patches can be printed individually or printed en masse and subsequently singulated using one of several common cutting techniques. A layer of polymeric binder compound, which is an insulative material when not combined with metallic particles, is preferably sprayed or printed over the patterned layer to seal the surface of the patterned layer. This will not only prevent the oxidation of any metallic particles that are exposed on the surface of the patterned layer, but will provide additional durability to the conductive particle layer. Finally, a thin, replaceable, non-allergenic adhesive layer is applied to the metalized side of the pain relief patch so that the patch can be adhered to the skin at or near the site of pain on a patient's body. Silicone adhesives are presently preferred for this application.

Referring now to FIG. 1, a first embodiment metalized pain-relief patch 100 has a kidney-shaped polyester-based TPU substrate 101 on which is printed a stylized, bilaterally-symmetrical metallic pattern 102 that radiates from a central arc 103 at the top of the dressing 100. It will be noted that all metallic elements of the pattern 101 are interconnected. A kidney-shaped substrate facilitates locating a pain-relief patch on the torso adjacent the neck.

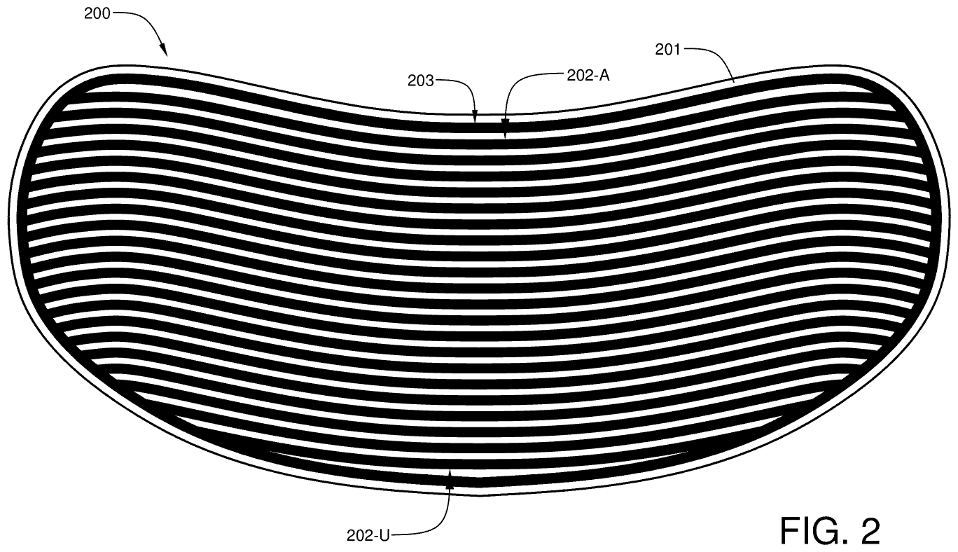

Referring now to FIG. 2, a second embodiment metalized pain-relief patch 200 has a kidney-shaped polyester-based TPU substrate 201 on which twenty-one parallel, evenly-spaced, curved longitudinal metallic traces (202, generally), which are sequentially numbered in the drawing 202-A to 202-U. All of the parallel traces 202-A through 202-U are interconnected at their ends by a circumferential metallic trace 203. It is presumed that the greater number of traces on a patch, the greater the probability that faint electrical signals under the skin will inductively couple with at least one trace 202. It is also presumed that inductive coupling will be more effective is the traces are aligned parallel to the current flow in nerve tissue.

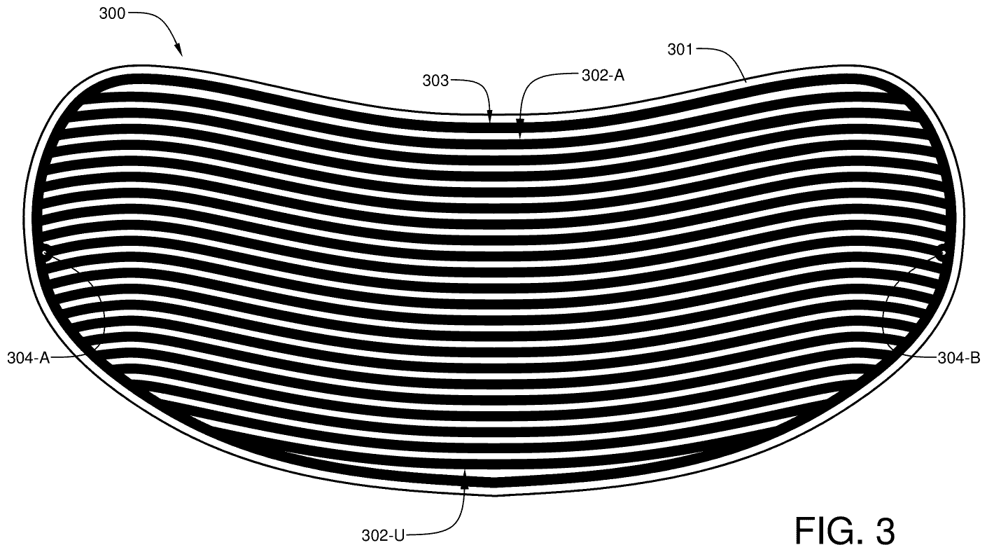

Referring now to FIG. 3, a third embodiment metalized pain-relief patch 300 is similar to the second embodiment metalized pain-relief patch 200 of FIG. 2. A kidney-shaped polyester-based TPU substrate 301 has been screen printed with twenty-one parallel, evenly-spaced, curved longitudinal metallic traces (302, generally), which are sequentially numbered in the drawing 302-A to 302-U. Also screen printed is a circumferential trace 303, which interconnects the ends of all longitudinal metallic traces 302. The difference is that there are two small punched apertures 303-A and 303-B at opposite ends of the dressing 300, which pierce the circumferential trace 302. It is through these two apertures 303-A and 303-B that contact will be made with a resistive trace on the opposite, or top, side of the dressing so that currents induced in traces 301-A to 301-U will be resistively dissipated and converted to heat.

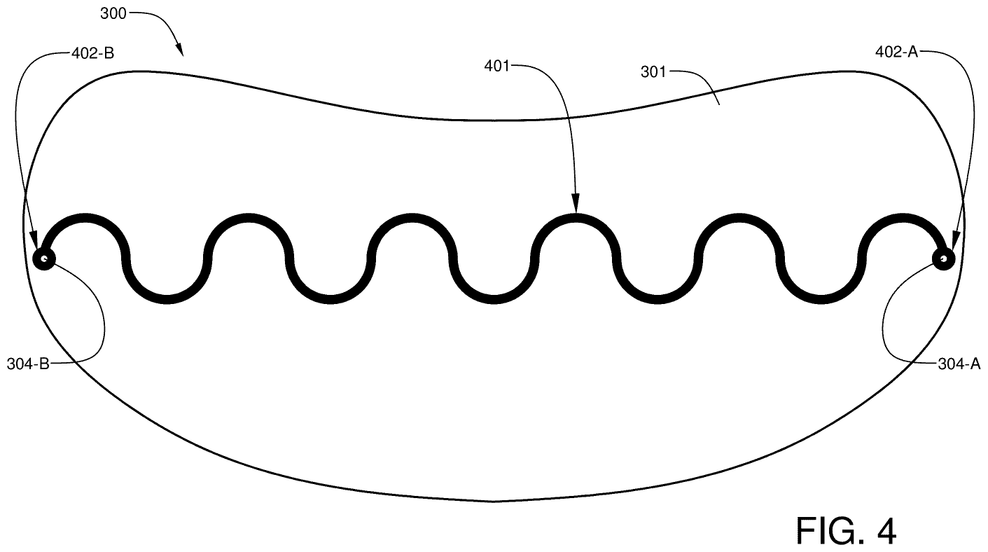

Referring now to FIG. 4, the top side of the third embodiment metalized pain-relief patch 300 of FIG. 3 is printed with a resistive graphite/carbon trace 401 that dissipates and converts to heat any currents flowing through the longitudinal traces 301 on the bottom side of the dressing 300. It will be noted that each end of the resistive trace 401 is equipped with a circular region 402-A or 402-B. These regions can be made larger or smaller depending on the accuracy of image registration with respect to the of the screen printing process, so that even with the misalignment inherent in the printing process, the punched apertures 303-A and 303B will be within the circular regions 402-A and 402-B, respectively. Alternatively, the two apertures 303-A and 303-B can be eliminated, and after printing an insulative layer over the patterned layer and leaving contact regions at opposite ends of the patterned layer, the resistive trace 401 may be printed on the same side of the dressing and making contact with the opposite ends of the patterned layer.

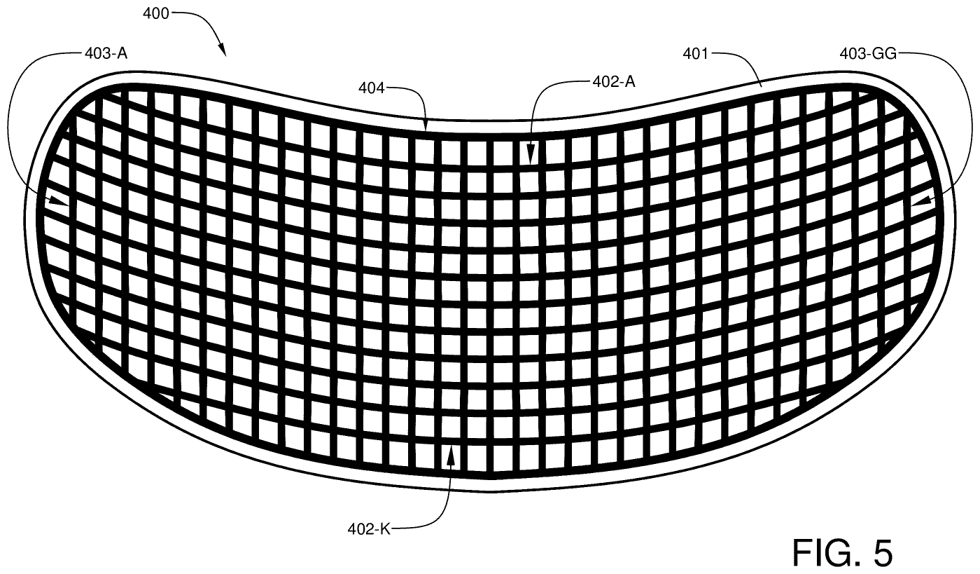

Referring now to FIG. 5, a fourth embodiment metalized pain-relief patch 400 has a kidney-shaped polyester-based TPU substrate 401 on which have been screen printed a an array formed by a grid consisting of eleven curved, parallel longitudinal metallic traces 402-A to 402-K, which intersect thirty-three straight, parallel transverse metallic traces 403-A to 403-GG. The grid is surrounded by a perimetric metallic trace 404 which is communication with the opposite ends of every other trace in the grid.

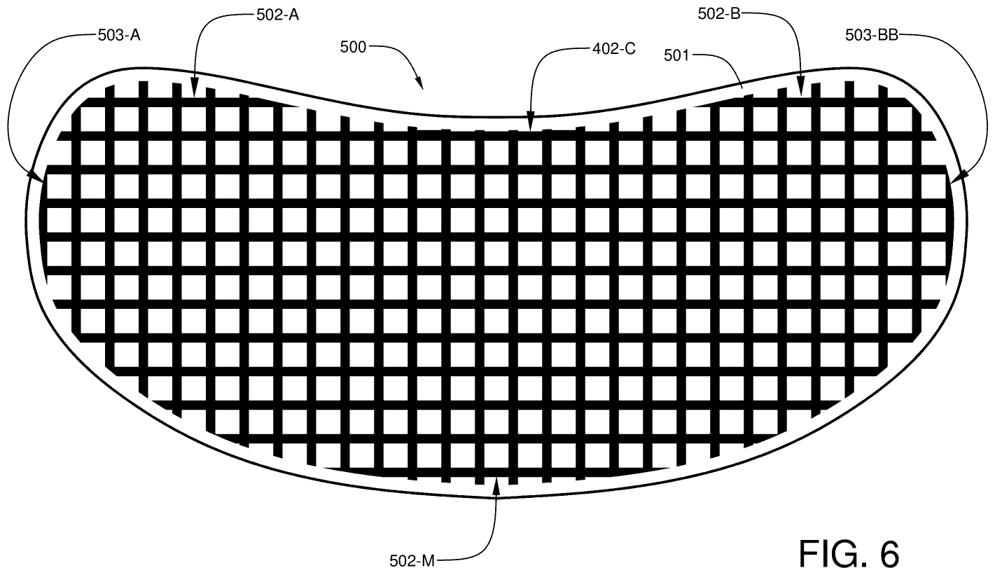

Referring now to FIG. 6, a fifth embodiment metalized kidney-shaped, metalized pain-relief patch 500 features a grid formed from a first set of parallel, evenly-spaced, straight longitudinal metallic traces 502-A to 502-M, which orthogonally intersect a second set of parallel, evenly-spaced, straight lateral metallic traces 503-A to 503-BB.

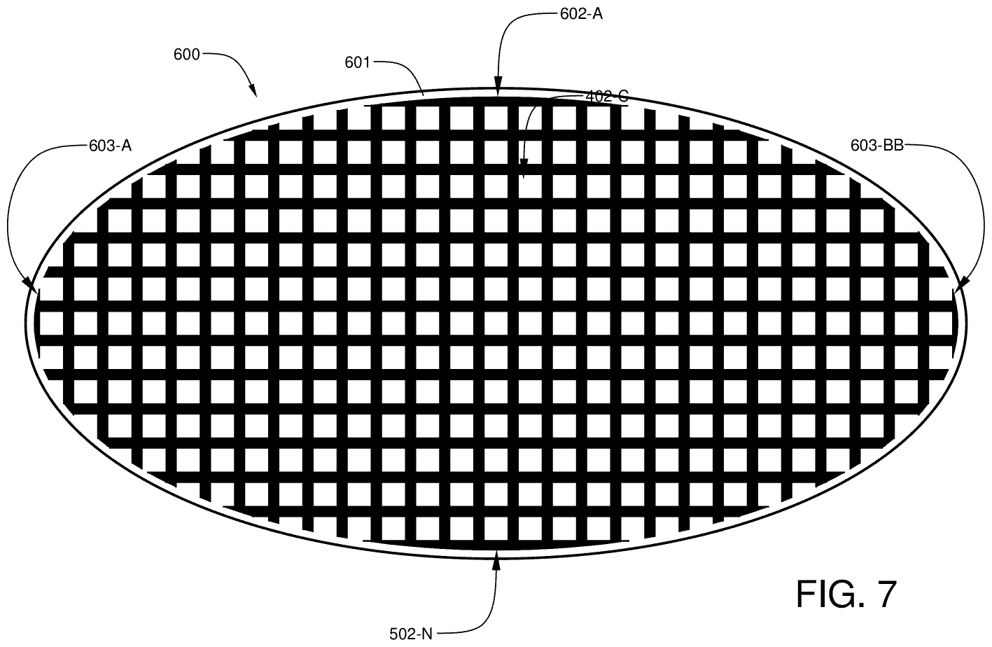

Referring now to FIG. 7, a sixth embodiment oval-shaped metalized pain-relief patch 600 features a grid formed from a first set of parallel, evenly-spaced, straight longitudinal metallic traces 602-A to 602-N, which orthogonally intersect a second set of parallel, evenly-spaced, straight lateral metallic traces 603-A to 603-BB.

Referring now to FIG. 8, an iteration of the second embodiment of the invention, which is a temporary conductive pattern tattoo 800 that is intended to be adhesively attached directly to a pain patient's skin at a site of pain. The broken conductive pattern 802 is, in this case, the same as the one used for the first iteration of the paint relief patch of FIG. 1. As a practical matter, any broken conductive pattern may be used. Fanciful broken conductive patterns showing the image of a in the image of dragon, a wolf, a lion, or an eagle, in addition to the ones shown in FIGS. 1, 2, 3, 5, 6 and 7, are but a few of the images which may be used for this application. The broken conductive pattern 802 is deposited on a decal backing paper sheet 801, using the screen printing process heretofore described for making the pain relief patches of FIGS. 1, 2, 3, 5, 6 and 7. Using the same pattern, a breathable, hypoallergenic or nonallergenic adhesive layer (not shown in this view) is deposited directly over the broken conductive pattern 802. Finally, a transparent release film is deposited over the entire temporary conductive pattern tattoo.

Referring now to FIG. 9, what is seen here is an ultra-thin slice 900, taken through the section line 9-9 of FIG. 8. By ultra-thin slice is meant that the slice has essentially no depth, and is intended to be merely illustrative of how the temporary conductive pattern tattoo 800 is fabricated. This ultra-thin slice definitely not drawn to scale, as that would be very difficult to do. The entire slice has a thickness of only about 0.008 inch (or about 0.2 mm), and the removable decal paper makes up about 0.003 inches (about 0.075 mm) of that total thickness. There are five layers in the ultra-thin slice 900: The bottom-most layer is the decal backing paper 901; the layer on top of the decal backing paper 901 is a water-soluble gum arabic layer 902; the broken-conductive pattern layer 903 is deposited on top of the gum arabic covered decal backing paper 901; a breathable hypoallergenic or nonallergenic adhesive layer 904 is deposited direcly on the adhesive layer 904; and a release film 905 covers the entire expanse of the decal backing paper 901. In order to apply the temporary conductive pattern tattoo 800 to a pain patient's skin, the patient peels off the release film, thereby exposing the adhesive layer 904. The patient then applies the temporary conductive pattern tattoo 800 (with the release film 905 removed), to his skin at the site of pain, with the adhesive layer 904 now in full contact with his skin. By applying pressure, with his hand, to the entire temporary conductive pattern tattoo 800, the temporary conductive pattern tattoo 800 becomes adhesively attached to his skin. The decal backing paper 901 can be removed by applying water thereto, which solvates the gum arabic layer, thereby allowing the decal backing paper 901 to be easily removed from the broken conductive layer, which is now exposed to the air and visible.

It is presently not fully understood why the patches and conductive pattern tattoos are effective at eliminating or mitigating pain. One theory is that the weak electric current in nerve tissue at the site of pain creates magnetic fields, which though also weak, can still interact with the metal layer of the patches through electromagnetic induction, thereby generating eddy currents in the conductive particle layer which are dissipated by the resistance to current flow within the conductive particle layer, itself. The electromagnetic induction and subsequent dissipation of the eddy currents in the conductive particle layer thereby interfere with electrical pain signals being sent to the brain by the body's nervous system. Another theory is that the conductive, broken layer, made of deposited conductive particles, acts as a first capacitor plate. A portion of a pain patient's body over which the pain patch lies, acts as a second capacitor plate. The adhesive layer, as well as the optional insulative layer printed on top of the conductive layer, provides an insulated gap between the first and second plates that creates the capacitor. If an electric charge differential exists between the two plates, electric field strength is certainly not uniform, as field strength presumably drops to zero where there are gaps in the conductive layer. It is possible that the variable electric field strength between the first and second capacitor plates somehow interferes with either the production of electrical pain signals, the attenuation of generated electrical pain signals, or the transmission of electrical pain signals that are produced by the body.

Although only several preferred embodiments of the metalized pain-relief patches for treating pain caused by injuries and disease have been shown and described herein, it will be obvious to those having ordinary skill in the art that changes and modifications may be made thereto without departing from the scope and the spirit of the invention as hereinafter claimed.

What is claimed is:

1. A pain-relief device comprising:
a flexible laminar substrate:
a broken conductive layer made of conductive particles in a polymeric binder matrix that is at least partially interconnected, both physically and electrically, and which is deposited on a first major surface of the flexible, laminar substrate;
a non-allergenic adhesive layer adhered to the broken conductive layer, said adhesive layer serving to adhere the broken conductive layer to a patient's skin at a site of pain; and
a release film, which covers the non-allergenic adhesive layer, as well as an entire major surface of the pain-relief device, and which can be subsequently peeled off and discarded so that the pain-relief device can be attached to a patient's skin at a site of pain.

2. The pain-relief device of claim 1, wherein the conductive particles are selected from the group consisting of silver-coated copper particles and powdered graphite.

3. The pain-relief device of claim 1, wherein the conductive particles in a polymeric binder matrix are deposited as a liquid, containing a volatile solvent, on the flexible substrate made of polymeric material, by means of a screen printing process using a screen having a mesh within a range of about 47 to about 165 threads per centimeter.

4. The pain-relief device of claim 2, wherein the flexible, laminar substrate is made of a polymeric material.

5. The pain-relief device of claim 4, wherein the polymeric material is polyester-based thermoplastic polyurethane (PET).

6. The pain-relief device of claim 2, wherein the flexible, laminar substrate is decal paper that has been coated with a gum arabic layer, and the broken layer of metal particles is deposited on top of the gum arabic layer.

7. The pain-relief device of claim 6, wherein the non-allergenic adhesive layer is deposited only on top of the broken layer of metal particles.

8. The pain-relief device of claim 7, wherein the decal paper coated with the gum arabic can be removed from the pain-relief device, once the release film has been removed and the pain-relief device adhered to a patient's skin, by simply wetting the decal paper and solvating the gum arabic layer so that the decal paper can be detached from the broken conductive layer, which remains on the patient's skin as a temporary broken conductive layer tattoo.

9. A pain-relief patch comprising:
a flexible laminar substrate made of polymeric material;
a broken conductive layer made of conductive particles in a polymeric binder matrix that is at least partially interconnected, both physically and electrically, and which is deposited on a first major surface of the flexible substrate;
a non-allergenic adhesive layer adhered to the broken conductive layer and to regions of the flexible substrate which are exposed by the broken conductive layer, said adhesive layer serving to adhere the pain-relief patch to a patient's skin at a site of pain; and
a release film, which covers the non-allergenic adhesive layer, as well as an entire major surface of the pain-relief patch, and which can be subsequently peeled off and discarded so that the pain-relief device can be attached to a patient's skin at a site of pain.

10. The pain-relief patch of claim 9, wherein the conductive particles are selected from the group consisting of silver-coated copper particles and powdered graphite.

11. The pain-relief patch of claim 9, wherein the polymeric material, from which the flexible laminar substrate is made, is polyester-based thermoplastic polyurethane (TPU).

12. The pain-relief patch of claim 9, wherein the conductive particles in a polymeric binder matrix are deposited as a liquid, containing a volatile solvent, on the flexible substrate made of polymeric material, by means of a screen printing process using a screen having a mesh within a range of about 47 to about 165 threads per centimeter.

13. The pain-relief patch of claim 9, wherein the conductive particles have diameters within a range of 5 to 13 microns.

14. A pain-relief, temporary broken conductive layer tattoo comprising:
a flexible substrate, in the form of a decal paper that has been coated with a gum arabic layer;
a broken conductive layer made of conductive particles in a polymeric binder matrix that is at least partially interconnected, both physically and electrically, and which is deposited on top of the gum arabic layer that coats the decal paper;
a non-allergenic adhesive layer adhered only to the broken conductive layer, said adhesive layer serving to adhere the broken conductive layer to a patient's skin at a site of pain; and
a release film, which covers the non-allergenic adhesive layer, as well as regions of the gum arabic coated decal paper that are exposed by gaps in the broken conductive layer, and which can be subsequently peeled off and discarded so that the pain-relief device can be attached to a patient's skin at a site of pain.

15. The pain-relief, temporary broken conductive layer tattoo of claim 14, wherein the conductive particles are selected from the group consisting of silver-coated copper particles and powdered graphite.

16. The pain-relief, temporary broken conductive layer tattoo of claim 14, wherein the conductive particles in a polymeric binder matrix are deposited as a liquid, containing a volatile solvent, on the gum arabic layer coated decal paper, by means of a screen printing process using a screen having a mesh within a range of about 47 to about 165 threads per centimeter.

17. The pain-relief, temporary broken conductive layer tattoo of claim 14, wherein the conductive particles have diameters within a range of 5 to 13 microns.

18. The pain-relief, temporary broken conductive layer tattoo of claim 14, wherein the release film is made of a transparent polymeric film.

19. The pain-relief, temporary broken conductive layer tattoo of claim 14, wherein once the temporary broken conductive layer tattoo has been adhered to a patient's skin, the decal paper can be removed from the broken conductive layer by wetting it, thereby solvating the gum arabic layer and thereby permitting it to be removed and discarded.

20. The pain-relief, temporary broken conductive layer tattoo of claim 14, wherein the conductive particles in a polymeric binder matrix are deposited as a liquid, containing a volatile solvent, on the flexible substrate made of polymeric material, by means of a screen printing process using a screen having a mesh within a range of about 47 to about 165 threads per centimeter.
